(19) United States
(12) Patent Application Publication
(10) Pub. No.: US 2002/0192717 A1
(43) Pub. Date: Dec. 19, 2002

(54) Cell-based assays for the simultaneous and discrete analysis of multiple analytes

(76) Inventors: Aaron B. Kantor, San Carlos, CA (US); Willem Peter Christiaan Stemmer, Los Gatos, CA (US); Remy Cromer, San Jose, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/104,476

(22) Filed: Mar. 22, 2002

Related U.S. Application Data
(60) Provisional application No. 60/278,269, filed on Mar. 23, 2001.

(51) Int. Cl. G01N 33/53; G01N 33/567; G01N 33/569; G01N 33/554

(52) U.S. Cl. 435/7.2; 435/7.31; 435/7.32

(57) ABSTRACT

Multiplexed immunoassays are performed using cells expressing on their surface capture agents such as antibodies or antibody fragments. The cells serve as the solid phase supporting the capture agent and also express identifiers encoding the nature of the capture agent, allowing the cells to be used in multiplexed assays. For example, the identifiers can be internally expressed fluorescent proteins or externally expressed proteins that bind to tagged antibody reagents. Analyte detection and quantification are performed by detection antibodies binding to bound analyte or by detection proteins expressed by the cell in response to analyte binding. By encoding capture, identification, and analyte detection functionalities within the cell, expensive and time-consuming steps of antibody preparation, purification, and coupling to a solid phase are eliminated, making the cells advantageous over antibody-coupled beads currently used in multiplexed immunoassays.

### CROSS-REFERENCE TO RELATED APPLICATIONS

This application claims priority from U.S. Provisional Application No. 60/278,269, “Cell-Based Assays for the Simultaneous and Discrete Analysis of Multiple Analytes,” filed Mar. 23, 2001, which is incorporated herein by reference.

### FIELD OF THE INVENTION

The present invention relates generally to methods for detecting analytes in biological samples. More particularly, it relates to methods and reagents for simultaneously and discretely detecting multiple analytes using multiple complementary binding moieties presented by distinguishable cell populations.

### BACKGROUND OF THE INVENTION

Quantitative assays, including immunoassays, are in widespread use for detecting and quantifying specific analytes in samples. Applications of such assays include medical diagnostics, biological research, and environmental testing. Analytes in solution can be measured quantitatively by employing a complementary binding moiety that is specific for the analyte, e.g., an antibody. Commonly, the complementary binding moiety is immobilized on a solid support that is then incubated with the analyte-containing solution. Various means can then be used to detect whether an analyte has been coupled to the binding moiety.

For example, in a sandwich immunoassay, anti-analyte “capture” antibodies are attached to a solid support such as a microtiter plate. The solid phase antibodies are incubated with a solution containing the analyte, washed, and then incubated with a second anti-analyte antibody. The second (“detection”) antibody ideally binds an epitope of the analyte that is distinct from the epitope to which the capture antibody binds. Typically, the detection antibody incorporates a tag to facilitate detection and quantification of the bound analyte. For assays performed in microtiter plate format, the detection signal, which can be absorbance, fluorescence, or radioactivity, or include enzymatic amplification of the signal, is read in bulk for each well. In general, the antibodies used in a sandwich immunoassay must be purified and chemically coupled to either the surface or the tag. Although arrays for performing such assays are in widespread use, they have a number of limitations, including lack of flexibility and small dynamic range, that have led to the development of alternative methods.

In solution array methods, the capture antibody is coupled to small, monodisperse particles, such as latex beads. The derivatized beads are incubated with a solution containing the analyte, washed, and then incubated with the detection antibody. As in the solid-phase assays, bound analyte can be detected in bulk. If the detection antibody has a fluorescent tag, the fluorescence intensity on each individual bead can be measured directly by flow cytometry, laser scanning cytometry, or imaging microscopy. These methods are described in M. J. Fulwyer and T. M. McHugh, “Flow Microsphere Immunoassay for the Quantitative and Simultaneous Detection of Multiple Soluble Analytes” chapter 51 in Methods in Cell Biology, vol. 33, eds. Z. Darzynkliewicz and H. A. Crissman, 1990, Academic Press, Inc., San Diego, pp. 613-629, incorporated herein by reference.

Latex beads can be used in multiplexed assays if beads containing different capture antibodies can be encoded differently. For example, U.S. Pat. No. 5,981,180, issued to Chandler et al., describes a multiplexed system in which beads are distinguished in real time by size, light scatter, or fluorescence intensity at one or more wavelengths. Typically, separate measurements are needed to identify the bead and quantify the bound analyte; for example, the detection antibody is tagged with a fluorophore of a different color from that attached to the bead. In this system, an entire bead set is incubated with a sample containing multiple analytes, washed, and incubated with a solution of detection antibodies. All of the detection antibodies can be tagged with the same fluorophore, because the detection antibodies are used for quantification and not for identification. Using a standard fluorescence imaging method, the analytes can be identified and quantified simultaneously.

One drawback of the multiplexed bead assay approach is the significant labor and time required to select and develop the antibody reagents, produce them in sufficiently large quantities, purify them, and couple them to the bead. Commonly, the beads themselves must be impregnated with different and specific levels of dye before the antibodies are bound to the beads. They are often also coated with a material that prevents non-specific binding of analytes to the beads, adding an additional preparation step. It would be highly beneficial to have a method for multiplexed solution-based assays that required fewer costly preparation steps.

Recently, methods have been developed for the expression of antibodies and other proteins on the surfaces of cells. U.S. Pat. No. 5,866,344, issued to Georgiou, incorporated herein by reference, discloses a method for creating antibody libraries by expressing antibodies on the surface of host cells such as bacterial cells. Antibodies that bind to analytes with high affinity can be selected by fluorescently (or otherwise) tagging analytes and separately combining them with antibody-bearing cells. The cells can also be used in quantitative competitive immunoassays, in which cells containing an antibody to an analyte of interest are combined with a sample suspected of containing the analyte and a tagged analyte at a known concentration. The concentration of unbound tagged analyte in the solution is proportional to the concentration of bound tagged analyte, and calibration curves can be constructed to aid in accurate analyte quantification. Although the cells cannot be distinguished from one another, the method of Georgiou allows for limited multiplexed assays by providing competitive analytes tagged with distinguishable tags (e.g., different colored fluorophores). According to this method, the same tags are used for analyte quantification and identification. Using the same signal to quantify analyte and identify the cell is disadvantageous because it decreases the system sensitivity; at low analyte concentrations, cells expressing antibodies for the analyte may not be detected at all, and there is no way to appropriately calibrate the low analyte concentration. Furthermore, each additional quantification tag must be optimized and calibrated, thereby limiting the multiplexing level permitted. The method of Georgiou provides for detection by measuring the fluorescence intensity of the supernatant, and not by counting the intensity of individual elements, as with bead-based methods.

A similar method for expressing antibodies and other proteins on the surface of yeast cells in a form accessible for binding to macromolecules is disclosed in U.S. Pat. No. 6,300,065, issued to Kieke et al., incorporated herein by reference. Yeast cells are preferable to prokaryotic expression systems such as bacteria for a number of reasons. Prokaryotic systems occasionally introduce unpredictable expression biases. Additionally, bacterial capsular polysaccharide layers present a diffusion barrier and may interfere sterically with macromolecular binding reactions. Yeast cells, in contrast to bacterial cells, have secretory pathways that are similar to those of mammalian cells, increasing the probability that proteins will fold correctly and be displayed on the surface. The method of Kieke et al. is used to select antibodies or other proteins from expression libraries based on their binding affinity or specificity. No methods are disclosed for performing multiplexed assays using these yeast cells.

There is still a need for reagents for performing multiplexed assays that can be produced with minimal antibody purification and preparation steps. Additionally, there is still a need for efficient methods for performing highly multiplexed immunoassays.

### SUMMARY OF THE INVENTION

The present invention provides methods and kits for performing multiplexed immunoassays using cells expressing proteins, such as antibodies, that bind to analytes of interest. The cells act as the solid phase supporting such “capture agents” and can be detected individually using standard methods and instruments such as flow cytometry, laser scanning cytometry, or fluorescence microscopy. Because the capture agents are expressed directly by the cells and do not require excessive purification and attachment steps, the cells can be produced in large quantities easily and quickly, in contrast to bead-based immunoassays.

A method of the invention for detecting at least one analyte is performed by providing a set of cells (or phage), each of which is characterized by a certain “cell identifier,” preferably an internal or surface-expressed polypeptide, and a surface-expressed capture agent such as an antibody, antibody fragment, or other protein. Preferable cells include bacteria (e.g., E. coli) or yeast. The capture agent is capable of binding to an analyte of interest in solution. Typically, the assay is performed by contacting the cell with a sample suspected of containing this analyte, and then detecting the cell identifier and assaying for, and possibly detecting, bound analyte. A “detection element” enables detection and quantification of bound analyte. Preferably, the detection element is a protein expressed internally by the cell in response to analyte binding to the capture agent, so that the amount of expressed detection protein indicates the amount of bound analyte. Alternatively, the detection element is secreted by the cell and binds to the bound analyte; it can also be provided to the assay as a separate reagent. Preferably, the detection element and cell identifier are fluorescent (or luminescent), allowing a standard method such as flow cytometry, laser scanning cytometry, or imaging microscopy to be employed. The method is typically performed in a multiplexed fashion by providing at least one additional cell type containing a different identifier and a capture agent capable of binding to a different analyte. All of the cells may be combined with the sample simultaneously, and the bound analyte and identifier of each cell are detected.

The present invention also provides a kit for performing multiplexed assays. The kit contains at least two types of cell, each cell type characterized by a different cell identifier and a different surface-expressed capture agent (e.g., antibody or antibody fragment) capable of binding to a different analyte. Preferable cell types are yeast and bacteria. Preferably, the cells also contain genetic vectors encoding detection proteins, which can be expressed in response to binding of analyte to the capture agent. The detection proteins can also be secreted by the cell for binding to the analyte. Alternatively, the kit can contain tagged detection antibodies that allow for detection and quantification of bound analyte, as well as identifier-binding antibodies that provide for identification of the cell type. The identifiers are typically proteins that are expressed internally or on the surface of the cells.

### BRIEF DESCRIPTION OF THE FIGURES

FIGS. 1A-1F are schematic diagrams of cells of the present invention.

FIG. 2 is a schematic diagram of a set of cells of the present invention for use in a multiplexed assay.

FIG. 3 shows a cytometry dot plot of identifier proteins and a histogram of detection proteins of each cell subset of FIG. 2.

### DETAILED DESCRIPTION OF THE INVENTION

The present invention provides reagents, kits, and methods for performing multiplexed assays such as immunoassays. Cells expressing complementary binding moieties, or “capture agents,” such as antibodies, antibody fragments, proteins, glycoproteins and other post-translationally-modified proteins, polypeptides, or aptamers, serve as the solid phase for sandwich, competitive, or other assays. Cell identifiers are used to label cells expressing particular binding moieties, allowing the cells to be used in multiplexed assays. In a standard flow or laser scanning cytometry system, detection and identification elements internal to a cell or tags on individual cell identifiers and bound analytes can be read, providing for very sensitive detection of a wide range of analyte concentrations.

Cell-based assays of the present invention provide a number of advantages over conventional multiplexed bead-based immunoassays. In preferred embodiments, all of the elements required for an assay (capture, detection, and identification) are encoded within a single cell. Once a particular assay is developed, subsequent cells require minimal time and cost to produce. A master cell population can be stored and individual populations grown from one of the master cells when needed. Cell-based assays have a high degree of reproducibility, since subsequent cells are essentially identical to the original ones. In contrast, for each new set of beads in a bead-based assay, capture agents must be produced, purified, and coupled to the beads, and variations can occur among different batches of antibodies and beads. Beads often require coatings to prevent non-specific binding of analytes, while appropriate cell strains can be selected or engineered to minimize non-specific binding in a particular sample matrix.

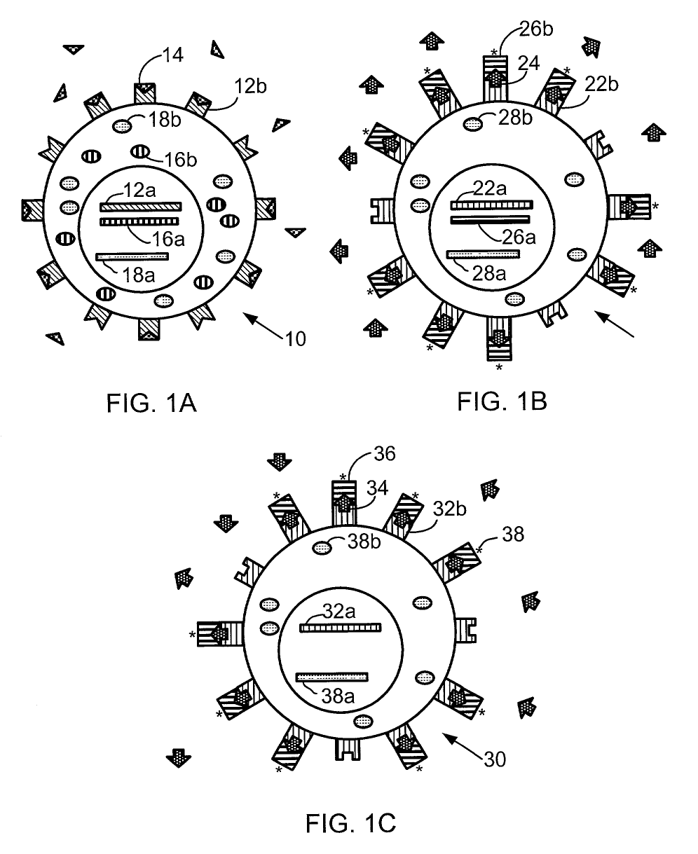

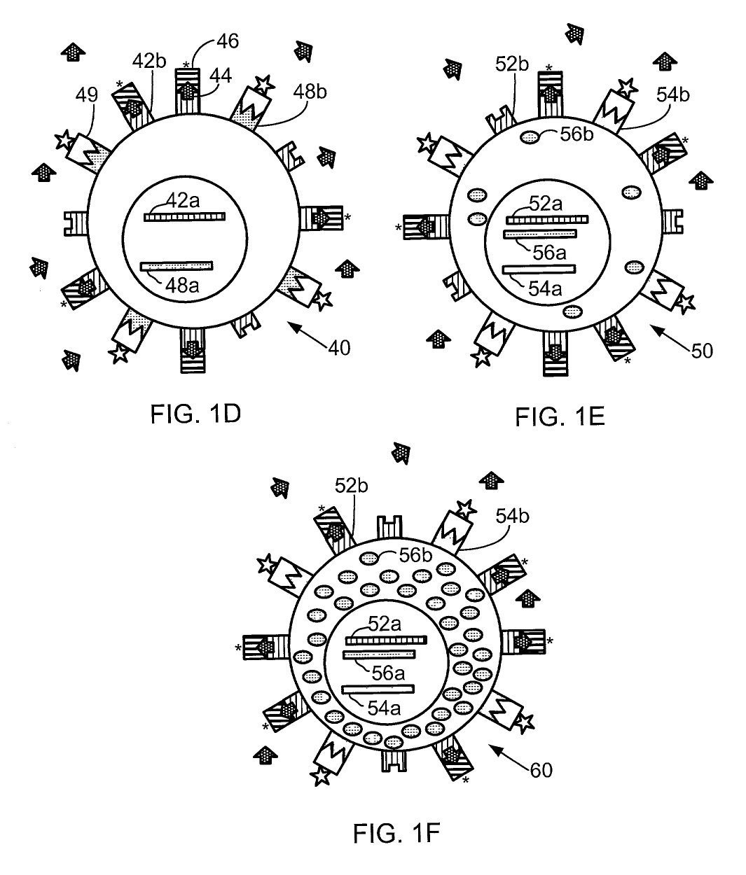

FIGS. 1A-1F illustrate different embodiments of cells of the present invention. Each cell type (also referred to as cell subset) of the present invention has the following three cell properties:

a capture agent or analyte-binding moiety expressed on its surface from a genetically encoded vector within the cell. Suitable capture agents include anti-analyte antibodies, antibody fragments, or other proteins or polypeptides.

a cell identifier expressed, either internally or externally, from a genetically encoded vector within the cell. The cell identifier—typically a protein or polypeptide—is used to identify cell types uniquely or distinguish cell subsets from each other.

means by which the amount of analyte bound to the capture agent can be determined via a detection element produced by the cell or added to the assay. Detection elements include proteins genetically encoded by inducible detection vectors and expressed internally or secreted in proportion to the amount of bound analyte, and tagged detection antibodies or other reagents.

These three cell properties implement the three assay elements: capture, detection, and identification.

FIG. 1A illustrates a preferred embodiment of a cell 10 of the present invention. The cell 10 is preferred to alternative embodiments because it provides all three assay elements, and additional reagents are not required to perform a multiplexed assay of the invention. The cell 10 contains a capture vector 12a expressing a capture agent 12b, such as an antibody, antibody fragment, or other protein or polypeptide, on the surface of the cell. The capture agent 12b is capable of specifically binding an analyte 14. The number of binding moieties or capture agents 12b expressed on each cell 10 is preferably large, ranging from on the order of 10³ to on the order of 10⁶ capture agents. A larger number of binding moieties (that do not interfere sterically) is preferable, because it increases the dynamic range of the system, i.e., allows for accurate determination of analyte quantities over a wide concentration range.

Also included in the cell 10 is a detection vector 16a containing an inducible reporter gene that is expressed following binding of the analyte 14 to the capture agent 12b. The detection vector 16a contains a promoter that regulates the amount of expression based on the level of analyte binding. The reporter gene expresses (internally or on the cell surface) a detection element, in particular a detection protein 16b that exhibits a detectable property such as fluorescence or luminescence. For example, the protein 16b can be green fluorescent protein, red fluorescent protein, or other similar reporter proteins used in gene expression monitoring (or fusion proteins containing such reporter proteins). The fluorescence intensities of these proteins are sufficient to allow their detection using standard flow cytometry, laser scanning cytometry, or fluorescence imaging instruments. The level of fluorescence or luminescence is measured to determine the amount of bound analyte 14. Preferably, the vector 16a has a linear response so that the fluorescence intensity is directly proportional to the amount of bound analyte, but any response for which a standard calibration curve can be constructed can be used.

Each cell 10 also contains an identifier vector 18a that expresses a cell identifier by which the nature of the capture agent is encoded, allowing for multiplexed assays. The cell identifier either has an intrinsic property that can be detected directly or can bind to or associate with a reagent having a detectable property. In both cases, this property is distinguishable (either in kind or degree) from that of the detection property used to detect and quantify the analyte. The cell identifier is independent from the expressed antibody serving as the binding moiety. The level of expression of the identifier preferably remains constant and is not affected by analyte binding to the capture agent. FIG. 1A illustrates one embodiment of a cell identifier 18b. In this case, the cell identifier 18b is an internally expressed protein or polypeptide with a detectable property such as fluorescence or luminescence. The identifier 18b is similar to the detection protein 16b but has a distinguishable property, e.g., a distinguishable fluorescence emission spectrum.

FIG. 1B illustrates an alternative embodiment, a cell 20, which is similar to the cell 10 but is used for performing sandwich immunoassays or other assays in which the analyte can bind to two distinct antibodies or other proteins simultaneously. Such assays require the analyte to have two distinct epitopes or other binding sites. In this embodiment, the cell 20 contains a vector 22a that expresses a capture agent 22b that binds to an analyte 24. The binding event can, but does not necessarily, regulate expression of a detection vector 26a. Rather than express an internal detection protein, the detection vector 26a expresses a protein 26b (e.g., a detection antibody) that is secreted from the cell and binds to a different site on the analyte 24. The detection protein 26b has, e.g., a fluorescent property that allows quantification of the amount of bound analyte. For example, the detection protein 26b can be a fusion of green fluorescent protein and the appropriate binding protein. Note that in this case, it is not necessary for the detection protein 26b to be expressed in proportion to the amount of binding. Rather, it is only important that enough detection protein 26b be secreted to bind to all of the bound analyte 24. If necessary, chemical switches of the type known in the art, triggered by the presence of a certain substance (e.g., alcohol or tetracycline), can be used to induce expression of the detection protein. Sandwich configurations provide higher specificity than single-capture agent assays and are therefore preferred in some particular applications. While there might be non-specific binding between an analyte and a single capture agent, it is much less likely that non-specific binding will cause both analyte binding and detection protein binding. In the sandwich configuration, even if an undesired analyte binds non-specifically, it is not detected if the detection protein does not bind to it. The cell 20 also contains an identifier vector 28a that expresses an internal identifier protein 28b.

As will be apparent to one of ordinary skill in the art, many variations of these two embodiments are possible, and all such variants are intended to fall within the scope of the present invention. For example, two forms of an antigen that bind to the same antibody can be distinguished by including vectors for two different secreted detection antibodies, each one binding to only one of the two antigen forms. The detection antibodies have distinguishable properties, e.g. different fluorescence emission spectra. The ratio of the two colors can be detected to determine the relative amounts of the two antigen forms, while the identity of the cells is determined by the identifer protein.

Note that embodiments of the present invention in which the cell contains vectors encoding all three assay elements (capture, identification, detection) can serve as biosensors. These multiplexed biosensors detect and indicate the presence and quantity of multiple analytes in contact with the sensors. In some cases, it is not necessary to quantify the amount of captured analyte, but rather only to indicate whether or not a particular analyte has been detected. The amount of detection protein is not regulated; expression is either on or off depending upon whether any analyte has bound to the capture agents. The biosensor can then be considered to have a simple YES or NO readout.

In alternative embodiments of the invention, additional reagents are required either for identification of the cell or for detection of the amount of bound analyte. The embodiments described below each include one implementation of each of the three different assay elements (capture, detection, identification). Although all possible combinations of these different implementations and those detailed above are not described explicitly, it is to be understood that every such possible combination is included in the scope of the present invention.

In FIG. 1C, a cell 30 contains a vector 32a that expresses on its surface an antibody 32b capable of binding to an analyte 34. A detection antibody 36 (the detection element) binds to a different epitope on the analyte 34 and contains a detection tag 38 such as a fluorescent tag. As with the previous embodiments, the detection tag 38 is used not for identification of the analyte 34 or antibody 36, but rather for quantification of the bound analyte 34. Unlike the previous embodiments, the cell 30 does not produce the antibody or other detection protein necessary to implement the detection mechanism. Rather, the detection antibody 36 or other detection element is provided as an additional reagent for detection. The configuration shown in FIG. 1C is useful for a sandwich immunoassay, but other types of assays (e.g., competitive) can be employed using appropriate reagents. Note that although this embodiment involves antibody production and purification, unlike the previous embodiments, the cell itself is simpler than cells 10 or 20. Depending upon the types of antibodies and reagents involved, one of the cells 20 or 30 may be preferred. The cell 30 also contains a vector 38a that expresses an internal identifier 38b such as green fluorescent protein.

FIG. 1D illustrates an alternative embodiment, a cell 40 containing an identifier vector 48a expressing an external identifier 48b. In this case, the cell identifier 48b is a protein or polypeptide that is expressed on the surface of the cell 40, in addition to the expression of the capture agent 42b by a capture vector 42a. In most cases, when the identifier 48b is expressed on the surface of the cell 40, it does not have an inherent property that can be detected easily using standard instrumentation. Instead, an additional tagged reagent is introduced to bind to the cell identifier for detection. For example, a fluorescently-tagged antibody reagent 49 can be introduced to bind to the identifier 48b; of course, the fluorescent tag for the identifier has a different emission spectrum from that of the fluorescent detection tag attached to the detection antibody 46 or other protein that binds to the analyte 44 captured by the capture agent 42b.

The cell identifiers allow different cell types or subsets of cells to be distinguished. The number of different identifiers that can be provided determines (in part) the level of multiplexing attainable using the present invention. One way to increase the degree of multiplexing is to combine multiple identifiers in a single cell. In this case, the identifier is a pattern of individual identifier proteins. FIG. 1E illustrates a cell 50 containing a capture vector 52a expressing a capture agent 52b, an external identifier vector 54a expressing an external identifier 54b, and an internal identifier vector 56a expressing an internal identifier 56b. Using a standard flow cytometer or laser scanning cytometer, the cell 50 can be distinguished from a cell containing only the external identifier 54b or only the internal identifier 56b. FIG. 1F illustrates an alternative embodiment of a cell 60 in which the ratio between internally and externally expressed identifiers is one factor distinguishing different identifiers. The cell 60 is similar to the cell 50 but a contains a much larger amount of the internally expressed identifier protein 56b, allowing the two cells 50 and 60 to be distinguished. As will be apparent to one of skill in the art, levels of internally or externally expressed proteins that can be distinguished depend upon a number of factors such as the intensity of the fluorescence or other property, detection techniques, and nature of the identifier. For example, a factor of ten or one hundred difference in expression is much easier to detect and produce reliably than a factor of two.

Note that although it is possible to use a single element for both detection (and quantification) of bound analyte and identification of the cell type, for a number of reasons, it is advantageous to have distinct identifier and detection elements as provided by the present invention. First, having distinct systems for detection and identification maximizes the assay sensitivity, because analyte quantification is performed on all cells that display identifiers, not just those with sufficiently high analyte level. Second, a single detection tag can be used for all analytes, requiring optimization of only that detection system. Because the fluorescence intensity of the identifier is not used quantitatively, it is not necessary to generate reliable calibration curves for the identifiers. It is only required that each cell produce sufficient identifier quantities to be detectable. Third, a higher degree of multiplexing is facilitated by keeping the detection and identifier systems distinct.

More complex cells with higher levels of functionality can be created using techniques developed in the field of cellular computation. In these methods, a cell serves as a biochemical computer, processing an input such as analyte binding using internal logic gates to generate an output. Complex conditional responses to multiple inputs have been engineered using AND, NOT, OR, XOR, and IMPLIES logic gates in E. coli cells. These gates are implemented using DNA-binding proteins to regulate expression of recombinant vectors. For more information on cellular computing, see R. Weiss, “Cellular Computation and Communications using Engineered Genetic Regulatory Networks,” Ph.D. Thesis, MIT, 2001; and M. L. Simpson et al., “Whole-cell biocomputing,” Trends Biotechnol. 19: 317-323 (2001), both of which are incorporated herein by reference. In the present invention, the output can be a signal proportional to the amount of analyte binding and nature of the analyte. A single plasmid can be created with different promoters switched on by different binding events to control expression of different detection proteins.

Cells of the present invention may be used in multiplexed assays. Multiple analytes of interest in a single sample can be detected simultaneously through their capture by different cell types or subsets. For example, an assay may be set up so that each cell type has a unique capture agent specific for an analyte of interest and a unique identifier (which can be a combination of identifiers) that identifies the capture agent. That is, there is a one-to-one relationship between the analyte-binding moiety and the cell identifier. The amount of analyte captured and identity of each cell are detected to determine the amount of each analyte in the original sample.

Note that the cells can also be used in assays for a single analyte or in multiple spatially separated assays. In this case, the identifier is used to confirm the identity of the cell type. This can be useful when multiple assays are performed in separate wells of a microtiter plate. Each well contains a different cell type, and the identity of the cell in each well is confirmed by detecting the cell identifier.

A variety of different types of assays (e.g., sandwich or competitive) can be performed using cells of the present invention. In general, the assay method follows a basic sequence of steps that may be modified by adding additional reagents as necessary. Additional reagents are added if the cell itself does not provide all three of the necessary assay elements (capture, detection, identification). Sandwich immunoassays, which can be performed using detection antibodies secreted by the cell or provided separately, are appropriate for analytes containing two distinct epitopes, each of which binds to a different complementary binding moiety (e.g., an antibody) in an independent manner. The epitopes should not overlap; that is, both antibodies should be able to bind the analyte at the same time without affecting the binding of the other. Furthermore, sandwich assays are generally more specific than assays in which the analyte binds to a single molecule only.

Competitive immunoassays are appropriate, for example, to detect analytes for which only one antibody or binding partner exists. Low molecular weight analytes typically can bind only one antibody at a time and cannot be detected using sandwich immunoassays. In a competitive immunoassay, a sample is combined with a known quantity of the analyte of interest bound to a tag such as a fluorescent tag. The tag is attached in such a way that it does not interfere with the binding of the analyte to the capture agent expressed on the surface of the cell. The amount of competitor reagent bound to the cell is proportional to the concentration of competitor reagent in solution, and therefore inversely proportional to the concentration of analyte in solution. Alternatively, the cell can express the analyte on its surface, and a tagged antibody is provided in solution as a competitor reagent. Free analyte in the sample and surface-bound analyte then compete for antibody binding sites, and the amount of antibody bound to the cell surface is inversely proportional to the analyte concentration in the sample. Cells expressing an internal detection protein in dependence on the amount of bound analyte may be used for performing assays that require neither a detection antibody nor a competitive reagent.

An assay of the present invention begins by contacting a collection of cell types (also referred to as cell subsets) with a sample such as a biological fluid sample. The sample is suspected of containing a number of different analytes of interest for which capture agents such as anti-analyte antibodies or other suitable complementary binding moieties are available. Each type of cell is specific for one of the analytes of interest, i.e., expresses on its surface a capture agent that can bind to one of these analytes. If a competitive assay is performed, the sample is combined with a known amount of tagged analytes or other suitable competitor reagent. A different competitor reagent is needed for each analyte suspected of being in the sample and its corresponding cell type. After exposure to the sample, the cells are washed to remove unbound material. Alternatively, the sample can simply be diluted.

If necessary, the cells are next exposed to additional reagents needed for detection of bound analyte or identification of the cell. In one embodiment, the additional reagents are tagged detection antibodies to the analytes suspected of being in the sample and potentially captured by the binding moieties in the previous step. The tagged detection antibodies bind with an epitope on the analyte that is distinct from the epitope to which the capture antibody binds. Alternatively, a substance such as alcohol or tetracycline is added to trigger production of the detection protein by the cell. In both cases, the detection antibody preferably has a fluorescent tag or is fused to a fluorescent protein, but other detectable properties such as radioactivity, chemiluminescence, or electrochemiluminescence can be used, with enzymatic signal amplification if desired. If any of the cells contain externally expressed identifier proteins, reagents allowing the detection of external identifiers are added. For example, the solution can contain fluorescently tagged anti-identifier antibodies containing fluorophores that have a different and distinguishable emission spectrum from that of the detection antibodies that bind to the captured analytes. If the cells produce internal identifiers and internal detection proteins, additional reagents are not required. The cells are then washed to remove unbound reagents, or alternatively simply diluted (which can decrease sensitivity).

The detection elements and identification proteins on or in each cell are assayed for and detected to quantify the bound analyte and to identify the cells. If the solution does not contain a particular analyte of interest, then the cells containing its capture agent are assayed and identified, but bound analyte is not detected. Fluorescence intensity on each cell can be read by methods known in the art such as flow cytometry, laser scanning cytometry, or imaging microscopy. In this way, the fluorescence intensity in all desired wavelength ranges on each individual cell can be detected. From this information, the analyte amount on each cell and the identity of each cell can be determined. The amount or concentration of analyte in the original sample can then be determined using standard methods.

Cells of the present invention are preferably made using one of a number of recently-developed techniques for expressing antibodies, antibody fragments, or other proteins or polypeptides on cell surfaces. In one technique, disclosed in U.S. Pat. No. 6,300,065, issued to Kieke et al., incorporated herein by reference, polypeptides are displayed on the surface of yeast cells by genetic fusion with cell wall proteins. Suitable yeast strains for this method include the genera Saccharomyces, Pichia, Hansenula, Schizosaccharomyces, Kluyveromyces, Yarrowia, and Candida. In the method of Kieke et al., yeast cells are transformed with a vector expressing the desired protein fused at its N-terminus to the C-terminus of a yeast cell wall binding protein, preferably to the binding subunit of the yeast agglutinin protein Aga2p. This protein is involved in cell-cell adhesion during yeast mating and is therefore exposed on the external surface of the cell. This ensures that the expressed binding moiety will be sterically accessible to the analyte in the assayed sample. Additionally, yeast secretory pathways are highly homologous to mammalian secretory pathways, indicating that active antibodies and other proteins can be folded, assembled, and expressed in yeast.

Alternatively, the protein can be expressed on the surface of bacterial cells such as E. coli using a method disclosed in U.S. Pat. No. 5,348,867, issued to Georgiou et al. Gram negative bacterial cells are preferred, but other suitable bacteria include Salmonella, Klebsiella, Erwinia, Pseudomonas aeruginosa, Haemophilus influenza, Rickettsia rickettsii, Neisseria gonorrhea, among others. In this method, a recombinant vector yields expression of the desired binding moiety as a fusion protein or polypeptide on the bacterial surface. The vector contains three elements: an anchoring sequence encoding a polypeptide capable of targeting and anchoring the fusion polypeptide to a host cell outer membrane, a membrane-traversing sequence encoding an amino acid sequence capable of transporting the polypeptide through the outer membrane, and the gene encoding the polypeptide that binds to the analyte (i.e., the capture agent). When provided with a functional promoter, the tripartite gene is expressible in gram-negative host cells.

Although these techniques have been developed for expressing antibodies and antibody fragments (e.g., single chain Fv antibody fragments), capture agents of the present invention include any polypeptides that can be expressed in a configuration accessible to analytes and that bind with sufficient affinity to analytes of interest. That is, any complementary binding moiety that can be expressed on the surface of a cell can be used. Additionally, the capture agent can bind to the analyte through an intermediate linker molecule, rather than directly. Known vectors for proteins with specificity for the analyte can be cloned into the cell type of choice, such as E. coli or yeast, using any available display technology such as phage display, ribosome display, puromycin-linked display, or any other display format in which the antibody or other capture agent is linked to the gene encoding it. In an alternative embodiment, the capture agent can instead be a nucleic acid such as DNA or RNA.

Alternatively, the capture agents and identifiers can be expressed on the surface of phage (e.g., filamentous, lambda, or T4), rather than host cells. Phage are advantageous because they are very stable and easily grown. The capture agents and identifier proteins are expressed as fusion proteins with the phage minor coat protein pIII or major coat protein pVIII. Approximately five proteins can be expressed fused to pIII and approximately 5400 fused to pVIII. Either the capture agents or identifier proteins can be fused to either coat protein. In most cases, detection requires an additional reagent to be introduced to bind to either captured analyte or the identifier protein. The identifier must be expressed externally, but an inherently fluorescent protein, such as green fluorescent protein, can be fused to the phage coat to serve as an identifier, without requiring an additional reagent. For more information on phage display, see G. P. Smith, “Filamentous fusion phage: Novel expression vectors that display cloned antigens on the virion surface,” Science 228: 1315-1317 (1985); A. S. Kang et al., “Linkage of recognition and replication functions by assembling combinatorial antibody Fab libraries along phage surfaces,” Proc. Natl. Acad. Sci. USA 88: 4363-4366 (1991); C. F. Barbas et al., “Assembly of combinatorial antibody libraries on phage surfaces: the gene III site,” Proc. Natl. Acad. Sci. USA 88: 7978-7982 (1991); and J. McCafferty et al., “Phage antibodies: Filamentous phage displaying antibody variable domains,” Nature 348: 552-554 (1990), all incorporated herein by reference.

Detection vectors such as the detection vector 16a of the cell 10 in FIG. 1A contain inducible reporter genes that are expressed following a binding event at the cell surface. These vectors contain promoters responsive to the binding of analyte to the capture agent. Examples of suitable inducible reporter genes include the gfp gene, which expresses green fluorescent protein, and the lacZ gene, which expresses β-galactosidase. In many cases, the expression level is either on or off. For the present invention, the expression of the detection protein (e.g., green fluorescent protein) is preferably correlated with the amount of analyte binding. Recently, Keasling et al. have developed regulated expression of the reporter genes lacZ and gfp under control of the arabinose promoter. In this system, the reporter gene expression is largely proportional to the concentration of arabinose. For more information on these systems, see A. Khlebnikov et al., “Regulatable arabinose-inducible gene expression system with consistent control in all cells of a culture,” J. Bacteriol. 182: 7129-7034 (2000); A. Khlebnikov et al., “Homogeneous expression of the P(BAD) promoter in Escherichia coli by constitutive expression of the low-affinity high-capacity AraE transporter,” Microbiology 147: 3241-3247 (2001); and C. D. Smolke et al., “Effects of transcription induction homogeneity and transcript stability on expression of two genes in a constructed operon,” App. Microbiol. Biotechnol. 57: 689-696, all incorporated herein by reference.

In the present invention, this system can be used to control expression of the detection protein in dependence on the level of analyte binding. A calibration curve is constructed by measuring the detection protein expression (i.e., its fluorescence) when the cells are combined with samples containing known concentrations of analyte. As long as a reproducible curve can be constructed, it is not necessary that the response be linear. The measured fluorescence intensity of the detection protein during an assay can then be correlated with the analyte concentration in the sample using the calibration curve.

The detection proteins can also be internally expressed luminescent proteins. Certain bacteria (e.g., Vibrio fischeri) have autoinducible luminescent genes that express luciferase, which causes cleaving of luciferin and emission of blue light. Bacteria produce signal molecules, N-acyl homoseine lactones (AELs), that enter bacterial cells and induce transcriptional activation of the genes LuxI, which encodes AHL synthetase, and LuxR, which encodes the AHL-dependent transcriptional activator. A sufficiently high concentration of AHL in the cell causes binding to the LuxR activator and transcription of the luminescence genes. In the present invention, binding of analyte causes expression of the luminescence genes in the host cells. The measured luminescence on each cell can be correlated with the amount of bound analyte.

Alternatively, the detection elements can be fusion proteins (e.g., green fluorescent protein-Fv) that have a detectable property and that are secreted from the cell and bind to the bound analyte. If the secretion of detection protein is independent of the level of analyte binding or related to a chemical switch triggered by, e.g., alcohol or tetracycline, then it is likely that sufficient detection protein is produced to bind to all bound analyte. It may be necessary to wash or dilute the cells sufficiently to minimize the background fluorescence from unbound detection protein. Alternatively, the secretion can be triggered by analyte binding to the capture agent, as with internally expressed detection proteins (described above). In this case, the detection element is produced in excess rather than in proportion to the analyte binding. It has been shown that secreted molecules can bind to antibodies on a cell surface; for example, in A. Scheffold et al., “Analysis and sorting of T cells according to cytokine expression,” Eur. Cytokine Netw. 9 (3 Suppl): 5-11 (1998), incorporated herein by reference, an affinity matrix method is described in which secreted cytokines are relocated to the cell surface by attachment to an artificially created antibody matrix.

Internal identifier proteins can be produced in the host cell using expression vectors encoding the identifier proteins. For example, vectors for expressing fluorescent proteins in green, red, yellow-green, and cyan are available as part of the Living Colors™ series of proteins from CLONTECH (Palo Alto, Calif.). These vectors can be added to cells expressing capture agents to yield fluorescent internal identifiers. The level of molecular expression can be controlled with strong and weak promoters, so that different expression levels (e.g., by factors of ten or one hundred) serve as different identifiers, thereby increasing the degree of multiplexing possible. Additionally, mutant gfp genes have been constructed that fluoresce between 20- and 35-fold more intensely than the wild type, as described in B. P. Cormack et al., “FACS-optimized mutants of the green fluorescent protein (GFP),” Gene 173: 33-36 (1996), incorporated herein by reference. The mutant proteins also fold more efficiently than the wild type when produced in E. coli, increasing the fluorescence difference versus that of the wild type to a factor of 100. See also A. Sacchetti et al., “Green Fluorescent Protein variants fold differentially in prokaryotic and eukaryotic cells,” J. Cell. Biochem. 81: 117-128 (2001), incorporated herein by reference. For the present invention, different mutant gfp genes can be selected to control the expression level for multiplexing purposes. The fluorescence generated by such fluorescent proteins can be detected by the same methods used to detect the fluorescent tags used for analyte quantification. Alternatively, vectors containing luminescence genes, as described above, can be added to the cells, and the luminescent signal used for cell identification. Internally expressed identifiers are advantageous because they do not require additional reagents for their detection or identification.

Internal identifiers can also be produced by introducing into the cell a membrane permeant such as carboxynaphthofluorescein diacetate. Such compounds can be loaded into live cells passively. Intracellular esterases hydrolyze the compound to yield a red fluorescent product. Similar products with different emission spectra can be used to achieve multiplexing. Different intracellular compartments can also be tagged by fluorescent compounds to serve as cell identifiers.

Externally-expressed identifiers typically lack natural fluorescence or other properties that make them easily detectable. Instead, additional reagents are introduced to bind to the identifiers to allow their detection and identification. Externally-expressed proteins are therefore selected based on their ability to bind with high affinity and specificity to antibodies or other detection elements. A large range of proteins fits this criterion, and any such protein can be used in the present invention. In fact, externally-expressed identifiers are advantageous for some applications because their large variety provides for very high levels of multiplexing. The vectors for cell surface identifiers can originate from intrinsic or extrinsic sources. For example, an important source of extrinsic vectors include mammalian genes incorporated into E. coli or yeast cells. For mammalian genes, yeast cells are more likely to yield proper folding, post-translational modification, and expression. Suitable external identifiers include CD antigens and MHC antigens, for which many fluorochrome-conjugated antibodies are commercially available. Another suitable example of surface-expressed identifiers is streptavidin, for which vectors are commercially available. Biotin-fluorophore conjugates are introduced to bind to the streptavidin identifiers. By conjugating biotin with different fluorophores and combining the conjugates with cell subsets before multiplexing, different identifiers can be obtained. The biotin-streptavidin interaction is of much higher affinity than most antibody-antigen interactions. Similarly, hormone receptors can be expressed at the cell surface and combined with hormone-fluorophore conjugates.

An alternative form of externally-expressed identifiers is those that are naturally expressed by the cells. Different strains of E. coli or yeast can be selected that express distinguishable forms of surface proteins. Monoclonal antibodies can be made to the selected strains and conjugated with fluorescent or other detection labels. Because of the large number of strains available, such identifiers provide for a high degree of multiplexing. Additionally, the same cell strain can be used for multiplexed assays if the antibodies are conjugated with different fluorophores and combined with different cell subsets before the cell subsets are combined. In this case, it is necessary to select an antigen-antibody pair that binds with high enough affinity that the antibody will remain bound during the assay.

Cells can also be coupled directly to receptors or ligands of interest. For example, cells can be chemically derivatized with streptavidin or other proteins that bind ligands with high affinity. The ligand or protein can be conjugated to fluorophores for multiplexing. Alternatively, the ligand can be conjugated to the cell and the corresponding receptor cross-linked to a fluorophore. Other suitable examples include hormones and hormone receptors, enzymes and irreversible inhibitors, aptamers and targets, or polynucleotide pairs such as DNA-DNA, DNA-RNA, or RNA-RNA. Fluorophores can also be chemically activated to react with cell surface amines, carboxylates, hydroxyl groups, phosphates, carbohydrates, or other suitable molecules. Phospholipid and fatty acid-fluorophore conjugates can label a cell by intercalating into the cell membrane.

In general, it is necessary to develop the assays for each particular application using a screening process. A number of cell populations are grown with different surface-expressed antibodies or other polypeptides, and populations having the desired antigen specificity are selected for use in the assays of the invention. Known vectors for proteins with specificity for the analyte of interest can be cloned into the desired cell type and strain. The capture agents, which can be monoclonal or polyclonal antibodies, can be produced by any available hybridoma or display technology, including phage display, ribosome display, puromycin-linked display, or any other display format in which the antibody or other protein is linked to the gene encoding it. The expression vectors are then introduced into the selected cells to generate cell populations displaying the protein expression library. Preferably, for a highly efficient selection process, a library containing at least one million members is expressed in the host cells. In some cases, the analyte of interest is not well characterized or well known, and providing a very large cell library allows selection of capture agents based on binding affinity rather than on structure of the capture agent.

Selection of capture agents for use in the assays occurs by combining the candidate cells with analytes of interest that have been tagged for detection, e.g., with a fluorescent material. The tagged analytes are combined with the cells displaying the antibody expression library under conditions that allow specific antigen-antibody (or other protein-protein) binding. Conditions can be varied to alter the level of analyte binding so that capture agents with high binding affinity can be identified. Cells to which fluorescently-labeled analytes are bound can be sorted using a conventional cell sorting method (e.g., fluorescence-activated cell sorting). The desired clones are selected and grown in large quantities to use for assays of the present invention. The identifier vector and detection vector can be added to the cells either after selection of the clones or before generating the candidate cells, in which case all of the potential cells express the identifier and detection protein. The cells can be further selected or engineered to minimize matrix effects, cross-reactivity, or other interfering effects from complex samples such as serum. The antigen specificity selection process is repeated for all analytes to be detected.

Preferably, both the analytes and the identifiers are detected by measuring fluorescence intensity. Fluorescent materials are commercially available and include small organic molecules such as fluorescein, rhodamine, and cyanine dyes; proteins such as phycoerythrin, allophycocyanin, and green fluorescent protein; tandem dyes such as Cy5-PE, Cy7-APC; and light-emitting semiconductor nanocrystals. Standard curves, obtained by titrating known amounts of the analyte, can be generated to facilitate quantification. For each analyte, measurement is made on multiple individual cells of a single type to afford sufficiently high signal-to-noise ratios and statistically significant results. For example, measurement is preferably made on at least ten, and preferably at least 100 individual cells of a single cell type. Cell concentrations range from approximately 10 to approximately 10⁴ per microliter.

As will be apparent to those of skill in the art, any other suitable assay detection technique such as radioimmunoassays, enzyme-linked immunoassays, or tags based on spectroscopic techniques such as surface-enhanced Raman scattering tags can be employed. These methods can be used for both cell identification (i.e., detection of the identifier protein's inherent property or its tag) and analyte detection and quantification. One suitable detection technique is fluorescence resonance energy transfer (FRET), which occurs between two neighboring fluorophores when the emission spectrum of one (the donor) overlaps the excitation spectrum of the second (the acceptor). The result of this exchange of energy is seen as a decrease in the specific emission intensity of the donor and an increase in the specific emission intensity of the acceptor. In particular, the fluorescence from the amino acid tyrosine can be quenched by nearby tryptophan residues. FRET occurs only when donor and acceptor fluorophore are in close proximity, typically <10 nm, allowing it to be used to indicate binding. In the present invention, the two amino acid fluorophores can be part of, e.g., the light and heavy chains of an antibody or the capture and detection antibodies. Binding of analyte to the antibody or to both capture agent and detection protein brings the donor and acceptor fluorophores in close enough proximity for FRET to occur. A similar phenomenon occurs when the two regions brought together by analyte capture are enzyme and substrate, producing a detectable product.

Enzyme-linked detection can also be used for analyte and identifier. For example, the identifier vector can contain the lacZ gene expressing the enzyme β-galactosidase, which hydrolyzes the fluorogenic and non-fluorescent substrate fluorescein di-β-D-galactopyranoside (FDG) to fluorescein monogalactoside and then to fluorescein. The fluorogenic substrate is added to the cell before the assay is performed. These kits are available commercially for flow cytometry use. Other fluorogenic substrates with different emission spectra provide for multiplexing capabilities.

In some specific cases, the analyte itself serves as the detection element. For example, the analyte can be an enzyme that, when bound to the capture agent, catalyzes a reaction in which a substrate is converted to a fluorescent or otherwise detectable product. In this case, neither a detection vector nor an external detection element is required.

The degree of multiplexing provided by the cells of the present invention depends upon the identifier and detection method employed, as well as the capture agents and analytes. As is known by those of skill in the art, analyte cross reactivities and matrix affects may limit the degree of multiplexing achievable for a particular assay. In the case of fluorescent tags, the degree of multiplexing depends in part upon the ability to distinguish the emission spectra of different fluorophores. Furthermore, the number of distinguishable identifiers can be increased by combining individual identifiers to form composite identifiers. For example, two independent identifiers, A and B, each fluorescing with different colors, allow for three different cell subsets to be identified, A, B, and AB, assuming that all cell subsets have at least one identifier. Similarly, three different identifiers, A, B, and C, allow seven different cell subsets: ABC, AB, AC, BC, A, B, and C. The degree of multiplexing is also affected by the detection methods; e.g., more fluorophores can be detected simultaneously using multiple laser excitation wavelengths. Using conventional fluorophores, nine different fluorophores can be detected and distinguished simultaneously using a three-laser system (M. Bigos et al., “Nine Color Eleven Parameter Immunophenotyping Using Three Laser Flow Cytometry,” Cytometry 36:36-45 (1999)). With a single laser, three fluorophores can be distinguished. The intensity level of the identifiers can also be modulated, allowing for between two and four distinguishable intensity levels (e.g., low, medium, high), making approximately nine different identifiers readily achievable. Multiplexing capabilities can be further increased by combining multiple types of tags, e.g., by combining fluorescent tags with non-fluorescent detection tags such as cell wall stains or colloidal gold particles, or including factors such as cell size. Additionally, time-resolved fluorescence can be employed by using fluorophores with long fluorescence lifetimes such as lanthanide chelates. Light-emitting semiconductor crystals (being developed by Quantum Dots Corporation, Hayward, Calif.), which can be detected in a standard flow cytometry instrument, may allow 6-10 different tags to be detected simultaneously with a single laser.

Cell-based assays of the present invention can be used for any application in which multiplexed immunoassays are required, such as diagnostic assays for multiple antigens known to be indicative of a particular disease or other physiological condition. One important application is differential phenotyping for biological marker discovery, in which a large number of analytes are detected simultaneously in a single biological sample. Biological markers, or biomarkers, are measured characteristics of a subject that are indicative of normal or pathological biological processes, response to therapy, or other clinical endpoints. By collecting samples from, e.g., healthy and diseased patients, drug responders and non-responders, or the same patients at different time points, differences in levels of particular analytes can be found that are indicative of the condition being investigated. While the large majority of measured analytes exist at comparable levels in both groups of subjects or time points, some analytes have statistically significantly different values between the two groups, and these analytes may serve as diagnostic or other biomarkers. In biomarker discovery studies, because the analyte of interest is unknown, it is important to be able to measure as many analytes as possible from a small sample volume so that relevant analytes or patterns of analytes can be identified. The present invention is advantageous for such multiplexed immunoassays because it provides capture agents that can be produced on a large scale with minimal reagent purification and processing steps, as well as extensive multiplexing capabilities.

The following example illustrates one possible implementation of a simultaneous cell-based immunoassay of the present invention. Table 1 lists the components of three different cell types used for a simultaneous assay for three different analytes of interest. Each cell type expresses one of three different capture vectors. Additionally, the first cell subset expresses both first and second internal identifier vectors, the second subset expresses only the first internal identifier, and the third subset expresses only the second internal identifier. All three subsets have an inducible vector expressing a fluorescent detection protein.

TABLE 1

| | Cell | Cell | Cell |
|---|---|---|---|
| Cellular components | Type 1 | Type 2 | Type 3 |
| ID vector1 (internal protein) | + | + | − |
| ID vector 2 (internal protein) | + | − | + |
| Capture vector 1 (anti-analyte 1 capture | + | − | − |
| antibody) | | | |
| Capture vector 2 (anti-analyte 2 capture | − | + | − |
| antibody) | | | |
| Capture vector 3 (anti-analyte 3 capture | − | − | + |
| antibody) | | | |
| Detection vector (internal protein) | + | + | + |

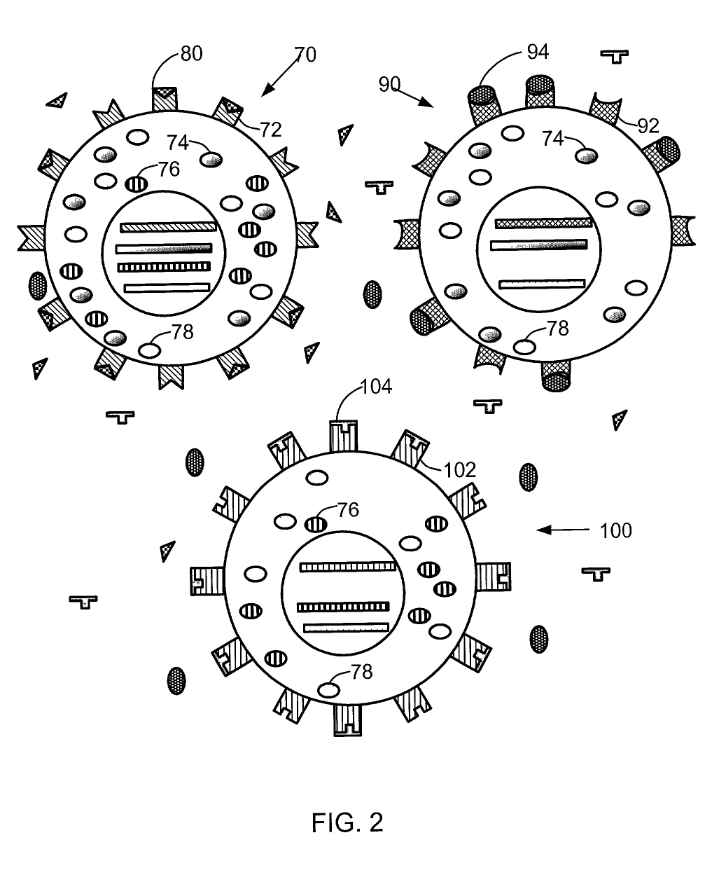

When these three cell types are used in an immunoassay, the cell collection is combined with the sample suspected of containing at least one of analytes 1, 2, or 3 and then washed or diluted. The resulting cells are shown schematically in FIG. 2. The first cell type 70 contains four vectors, one each for a capture antibody 72, a fluorescent first internal identifier 74, a fluorescent second internal identifier 76, and a fluorescent detection protein 78. After the assays are complete, the first cell type 70 has bound to it a first analyte 80. The second cell type 90 contains three vectors, one each for a second capture antibody 92, the first identifier 74 (same as on cell type 70), and the detection protein 78. After the assays, the second cell type 90 has bound to it a second analyte 94. The third cell type 100 contains three vectors, one each for a capture antibody 102, the second internal identifier 76, and the detection protein 78. After the assays, the cell type 100 has bound to it a third analyte 104. In these assays, no additional reagents are required. In FIG. 2, the complementary shapes indicate the specificities of each antibody for its respective antigen.

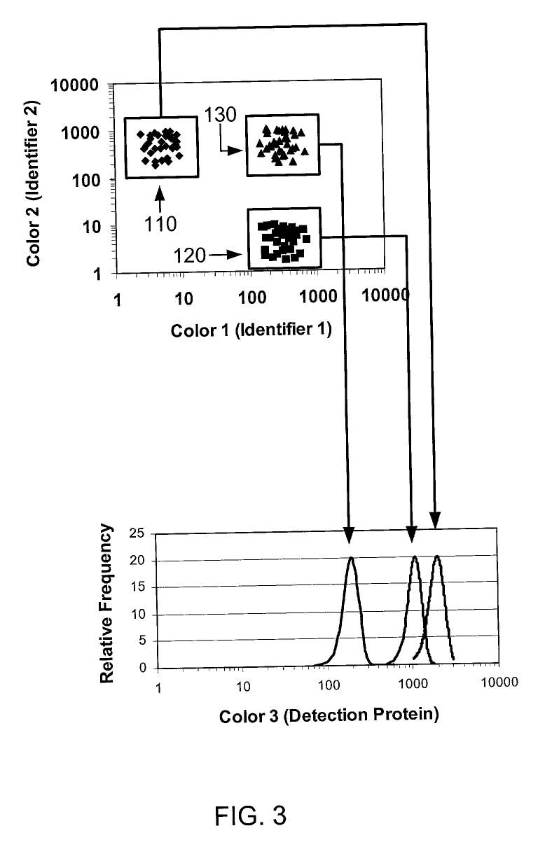

In order to identify and quantify the captured analyte, three different fluorescence intensities must be monitored, one for the detection protein and one for each of the two internal identifiers. FIG. 3 shows on the top a schematic cytometry dot plot for the cells of FIG. 2 obtained using a flow cytometer or microvolume laser scanning cytometer. Each point in the plot represents the fluorescence intensity in two colors of a single cell. The two colors are the colors associated with the two identifiers. The cells cluster into three regions of the plot, cluster 110 corresponding to only the color of the second internal identifier, representing cell type 100; cluster 120 corresponding to only the first color of the first internal identifier, representing cell type 90; and cluster 130 to the cells having both internal identifiers, cell type 70. Standard cytometry software allows each cluster to be selected for analysis as indicated by the boxes (or gates) shown. In the plot on the bottom, the frequency of occurrence of intensity of the third color in a single cell is shown for each cell type. The third color corresponds to the fluorescence of the detection protein. From this plot, the concentration of the three analytes in the sample can be determined.

It should be noted that the foregoing description is only illustrative of the invention. Various alternatives and modifications can be devised by those skilled in the art without departing from the invention. Accordingly, the present invention is intended to embrace all such alternatives, modifications and variances which fall within the scope of the disclosed invention.

What is claimed is:

1. A method for assaying for at least one analyte, comprising:
a) providing a set of cells, each comprising a cell identifier and a surface-expressed capture agent capable of binding to said analyte;
b) contacting said set of cells with a sample suspected of containing said analyte;
c) assaying for analyte bound to said set of cells; and
d) detecting said cell identifiers.

2. The method of claim 1, further comprising:
providing a second set of cells, each comprising a second cell identifier and a second surface-expressed capture agent capable of binding to a second analyte;
contacting said second set of cells with said sample;
assaying for analyte bound to said second set of cells; and
detecting said second cell identifiers.

3. The method of claim 1, wherein said capture agent is selected from the group consisting of an antibody and an antibody fragment.

4. The method of claim 1, wherein said cells are selected from the group consisting of yeast and bacteria.

5. The method of claim 1, wherein assaying for analyte bound to said set of cells comprises assaying for a detection protein produced by said cells.

6. The method of claim 5, wherein said detection protein is produced in response to binding of said analyte to said capture agent.

7. The method of claim 5, wherein said detection protein is an internally expressed protein.

8. The method of claim 5, wherein said detection protein is a secreted protein capable of binding to said analyte.

9. The method of claim 1, wherein assaying for analyte bound to said set of cells comprises detecting fluorescence intensity.

10. The method of claim 9, wherein said fluorescence intensity is detected using a method selected from the group consisting of flow cytometry, laser scanning cytometry, and imaging microscopy.

11. The method of claim 1, wherein said cell identifier is a polypeptide expressed by said cells.

12. The method of claim 11, wherein said polypeptide is expressed internally.

13. The method of claim 11, wherein said polypeptide is expressed on the surface of said cells.

14. The method of claim 1, wherein detecting said cell identifier comprises detecting fluorescence associated with said cell identifier.

15. A method for assaying for at least two analytes simultaneously, comprising:
a) providing at least two types of cells, each comprising a different cell identifier and a surface-expressed capture agent capable of binding to a different analyte;
b) contacting said cells with a sample suspected of containing at least one of said analytes;
c) assaying for analyte bound to each cell; and
d) detecting said cell identifiers on each cell.

16. The method of claim 15, wherein said capture agents are selected from the group consisting of antibodies and antibody fragments.

17. The method of claim 15, wherein said cells are selected from the group consisting of yeast and bacteria.

18. The method of claim 15, wherein assaying for analyte bound to each cell comprises detecting a detection protein produced by said cell.

19. The method of claim 18, wherein said detection protein is produced in response to binding of one of said analytes to one of said capture agents.

20. The method of claim 18, wherein said detection protein is an internally expressed protein.

21. The method of claim 18, wherein said detection protein is a secreted protein capable of binding to one of said analytes.

22. The method of claim 18, wherein assaying for analyte bound to each cell comprises detecting fluorescence intensity.

23. The method of claim 22, wherein said fluorescence intensity is detected using a method selected from the group consisting of flow cytometry, laser scanning cytometry, and imaging microscopy.

24. The method of claim 18, wherein said cell identifiers are polypeptides expressed by said cells.

25. The method of claim 24, wherein said polypeptides are expressed internally.

26. The method of claim 24, wherein said polypeptides are expressed on the surface of said cells.

27. The method of claim 18, wherein assaying for said cell identifiers comprises detecting fluorescence associated with said identifiers.

28. A kit for performing multiplexed assays, comprising at least two types of cells, each cell type comprising a different cell identifier and a surface-expressed capture agent capable of binding to a different analyte.

29. The kit of claim 28, wherein said capture agent is selected from the group consisting of an antibody and an antibody fragment.

30. The kit of claim 28, wherein said cells are selected from the group consisting of yeast and bacteria.

31. The kit of claim 28, wherein at least one of said cell types further comprises a genetic vector encoding a detection protein.

32. The kit of claim 31, wherein said detection protein is a fluorescent detection protein.

33. The kit of claim 31, wherein said detection protein is expressed in response to binding of one of said analytes to one of said capture agents.

34. The kit of claim 31, wherein said detection protein is secreted from said cell and is capable of binding to one of said analytes.

35. The kit of claim 28, further comprising tagged detection proteins capable of binding to at least one of said analytes.

36. The kit of claim 28, wherein said cell identifier is a polypeptide expressed by said cell.

37. The kit of claim 36, wherein said polypeptide is expressed internally.

38. The kit of claim 37, wherein said polypeptide is fluorescent.

39. The kit of claim 36, wherein said polypeptide is expressed on the surface of said cell.

40. A method for assaying for at least two analytes simultaneously, comprising:
a) providing at least two types of phage, each comprising a different surface-bound identifier and a surface-bound capture agent capable of binding to a different analyte;
b) contacting said phages with a sample suspected of containing at least one of said analytes;
c) assaying for analyte bound to each phage; and
d) detecting said identifiers on each phage.
